(19) United States
(12) Patent Application Publication
(10) Pub. No.: US 2024/0404623 A1
(43) Pub. Date: Dec. 5, 2024

(54) SYSTEM AND METHOD FOR PERSONALIZED INTERPRETATION OF GENETIC VARIANTS

(71) Applicant: The Royal Institution for the Advancement of Learning/McGill University, Montreal (CA)

(72) Inventors: John TRAKADIS, Mount Royal (CA); Lin Qi, Montreal (CA); Sameer SARDAAR, Montreal (CA)

(73) Assignee: The Royal Institution for the Advancement of Learning/McGill University, Montreal (CA)

(21) Appl. No.: 18/679,013

(22) Filed: May 30, 2024

Related U.S. Application Data
(60) Provisional application No. 63/505,175, filed on May 31, 2023.

(51) Int. Cl. G16B 20/00; G06N 3/042; G16B 15/00; G16B 40/20; G16B 45/00

(52) U.S. Cl. CPC G16B 20/00; G06N 3/042; G16B 15/00; G16B 40/20; G16B 45/00

(57) ABSTRACT

Methods and systems for phenotyping based on a neural network model are provided. Training the model includes generating a dataset of individual-specific variant graphs associated representing dependencies among variants in individuals' genomes based on biomedical domain knowledge labelled according to target phenotype values to be predicted, and training the model using the training dataset to generate a phenotype prediction from an input individual-specific variant graph. Measure the contribution of a genetic variant to a phenotype of a target individual includes generating an individual-specific variant graph for the individual, modifying the individual-specific variant graph to remove a genetic variant, providing the initial and the modified individual-specific variant graphs as input to the phenotyping neural network model to generate two phenotype predictions, and calculating a difference between the two predictions, providing a measure of the contribution of the genetic variant to the phenotype.

### CROSS-REFERENCE TO RELATED PATENT APPLICATION

This application claims the benefit of, and priority to, United States Provisional Patent Application No. 63/505,175, filed 31 May 2023, and entitled “System and Method for Personalized Interpretation of Genetic Variants”, the disclosure of which is hereby incorporated by reference in its entirety.

### TECHNICAL FIELD

The technical field relates to phenotyping, and more specifically to systems and methods for providing a personalized interpretation of a person's generic variants.

### BACKGROUND

Genetic variants are changes to the DNA sequence of an individual. As the amount of sequencing data grows, so does the number of novel genetic variants found and it is often challenging to understand the effects of these variants on the health status vs. disease. Current approaches to genetic variant interpretation follow the ACMG guidelines to classify variants into five discrete categories ranging from benign to pathogenic (disease-causing). The current guidelines focus on single-gene disorders and do not have clinical application for multifactorial diseases. Moreover, the variant classifications are variant-specific rather than patient-specific. Hence, the current paradigm cannot explain the impact of a variant in the context of a specific individual with a unique genomic background and so it cannot address challenges such as incomplete penetrance and variable expressivity for single gene and/or multifactorial diseases.

There remains a need for more robust approaches for personalized interpretation of genetic variants that take into consideration the sequence and attribute information about variants, interactions between variant and other variants in the genomic background of a given individual, as well as biological knowledge and patient phenotype.

### SUMMARY

In accordance with an aspect, a method for phenotyping is provided. The method includes generating a plurality of individual-specific variant graphs, each individual-specific variant graph being associated with an individual and representing dependencies among variants in a genome of the individual based on biomedical domain knowledge, generating a training dataset including at least some of the plurality of individual-specific variants graphs labelled according to corresponding target phenotype values to be predicted, and training a phenotyping neural network model using the training dataset to generate a phenotype prediction from an input individual-specific variant graph.

In accordance with another aspect, a method for phenotyping is provided. The method includes generating an initial individual-specific variant graph for a target individual, the initial individual-specific variant graph representing dependencies among variants in a genome of the target individual based on the biomedical domain knowledge, providing the initial individual-specific variant graph as input to the phenotyping neural network model, thereby generating a first phenotype prediction, modifying the initial individual-specific variant graph to remove a genetic variant, thereby generating a modified individual-specific variant graph, providing the modified individual-specific variant graph as input to the phenotyping neural network model, thereby generating a second phenotype prediction, and calculating a difference between the first phenotype prediction and the second phenotype prediction, the difference providing a measure of a contribution of the genetic variant to a phenotype of the target individual.

In accordance with a further aspect, a system for phenotyping is provided. The system includes one or more processor, memory having a phenotyping neural network model stored thereon, and a training module. The training module is configured to cause the one or more processor to generate a plurality of individual-specific variant graphs, each individual-specific variant graph being associated with an individual and representing dependencies among variants in a genome of the individual based on biomedical domain knowledge, generate a training dataset including at least some of the plurality of individual-specific variants graphs labelled according to corresponding target phenotype values to be predicted, and train the phenotyping neural network model using the training dataset to generate a phenotype prediction from an input individual-specific variant graph.

In accordance with yet another aspect, a system for phenotyping is provided. The system includes one or more processor, memory having a phenotyping neural network model stored thereon, and an inference module. The inference module is configured to cause the one or more processor to generate an initial individual-specific variant graph for a target individual, the initial individual-specific variant graph representing dependencies among variants in a genome of the target individual based on biomedical domain knowledge, provide the initial individual-specific variant graph as input to the phenotyping neural network model, thereby generating a first phenotype prediction, modify the initial individual-specific variant graph to remove a genetic variant, thereby generating a modified individual-specific variant graph, provide the modified individual-specific variant graph as input to the phenotyping neural network model, thereby generating a second phenotype prediction, and calculate a difference between the first phenotype prediction and the second phenotype prediction, the difference providing a measure of a contribution of the genetic variant to a phenotype of the target individual.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment.

FIG. 1 is a flowchart of a method for training a phenotyping neural network model and measuring the contribution of a genetic variant to a phenotype in a target individual according to an embodiment.

FIGS. 2A to 2C are plots showing the receiver operating characteristic curve for the prediction of a common disease class by two reference implementations (FIGS. 2A and 2B) and an implementation based on an embodiment disclosed herein (FIG. 2C).

FIGS. 3A and 3B are plots showing the contribution of genetic variants to a disease prediction for two individuals based on an embodiment described below.

FIG. 4A is a schematic illustrating a system for training a phenotyping neural network, according to an embodiment.

FIG. 4B is a schematic illustrating a system for obtaining a measure of the contribution of a given genetic variant to the phenotype of an individual using a phenotyping neural network, according to an embodiment.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practised without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

One or more systems described herein may be implemented in computer program(s) executed on processing device(s), each comprising at least one processor, a data storage system (including volatile and/or non-volatile memory and/or storage elements), and optionally at least one input and/or output device. “Processing devices” encompass computers, servers and/or specialized electronic devices which receive, process and/or transmit data. As an example, “processing devices” can include processing means, such as microcontrollers, microprocessors, and/or CPUs, or be implemented on FPGAs. For example, and without limitation, a processing device may be a programmable logic unit, a mainframe computer, a server, a personal computer, a cloud-based program or system, a laptop, a personal data assistant, a cellular telephone, a smartphone, a wearable device, a tablet, a video game console or a portable video game device.

Each program is preferably implemented in a high-level programming and/or scripting language, for instance an imperative e.g., procedural or object-oriented, or a declarative e.g., functional or logic, language, to communicate with a computer system. However, a program can be implemented in assembly or machine language if desired. In any case, the language may be a compiled or an interpreted language. Each such computer program is preferably stored on a storage media or a device readable by a general or special purpose programmable computer for configuring and operating the computer when the storage media or device is read by the computer to perform the procedures described herein. In some embodiments, the system may be embedded within an operating system running on the programmable computer.

Furthermore, the system, processes and methods of the described embodiments are capable of being distributed in a computer program product comprising a computer readable medium that bears computer-usable instructions for one or more processors. The computer-usable instructions may also be in various forms including compiled and non-compiled code.

The processor(s) are used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory is, of course, chosen according to the desired use, whether it should retain instructions, or temporarily store, retain or update data. Steps of the proposed method are implemented as software instructions and algorithms, stored in computer memory and executed by processors.

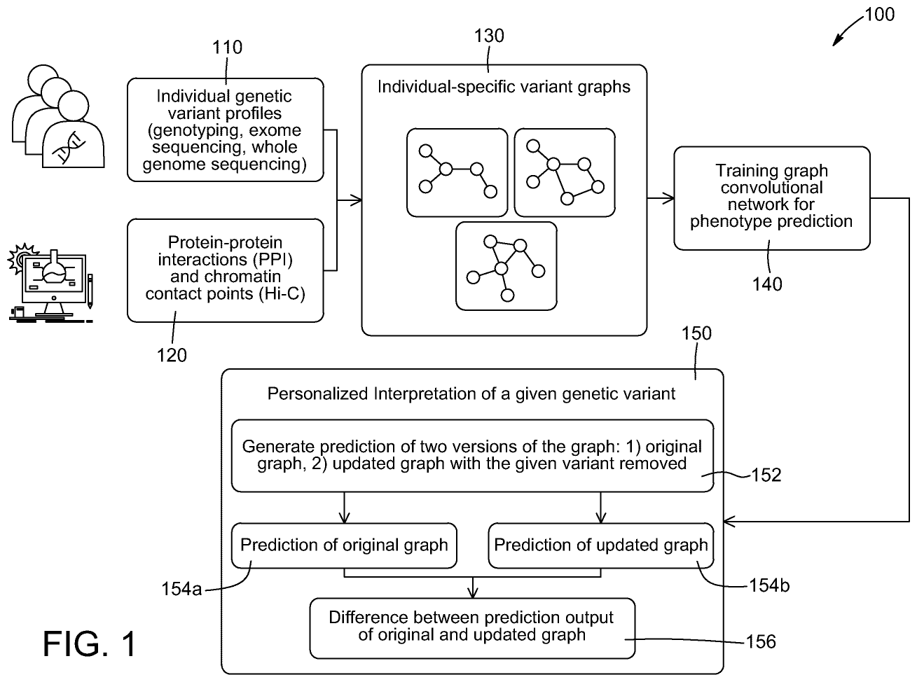

With reference to FIG. 1, an exemplary method 100 for training a phenotyping neural network model and measuring the contribution of a genetic variant to a phenotype in a target individual is shown. The method 100 can be at least partially carried out on a system for personalized interpretation of genetic variants, including memory and one or more processors. Broadly described, method 100 provides personalized interpretation of genetic variants by considering the genetic interactions between variants in an individual's genome. This helps to better decipher the potential effect of a given genetic variant in the context of other genetic variants in the individual's genomic background for a given phenotype, such as overall health or a specific disease. The method 100 uses graph representations of genetic variants based on biomedical domain knowledge, such as protein-protein-interaction (PPI) and chromosome contact (Hi-C) information. This knowledge is combined with an individual's genetic variant profile at step 130 to create a graph capturing potential genetic interactions, context, and proximity information among the variants of the given individual. Data-driven graph neural network models trained at step 140 are used to model the contribution of genetic variants to the desired phenotype. To obtain personalized interpretation of a genetic variant, a method based on counterfactual reasoning is used at step 150 to determine how the prediction would change when the variant is removed from the graph.

Genetic variants are changes to the DNA sequence of an individual. There are several types of changes that can occur, including single nucleotide variation, insertions, and deletions, as well as structural variations. Genetic variants can result in changes in the downstream transcription of genes, subsequent translation into proteins, and manifest as various phenotypes or diseases. On average, there is a genetic difference of about 0.1% of DNA between any pair of human individuals. Currently, there are over 900 million common genetic variations reported based on 192 k subjects from the ALFA (Allele Frequency Aggregator) project.

Each variant can be characterized by a number of attributes, such as its location on a particular chromosome, whether it occurs in a protein-coding gene or in an intergenic region between genes, the specific gene it is located in, whether it leads to a synonymous, non-synonymous, stop-gain, or stop-loss mutation in a protein-coding gene, its frequency in affected and unaffected populations, and various in silico predictions of its potential effects. These attributes, along with clinical and family history (including whether the variant is a de novo mutation or inherited from a parent, and its presence in affected or unaffected family members) are used to determine whether a variant is disease-causing (pathogenic) or benign.

In general, variant classification as pathogenic or benign is independent of an individual's genomic background. The current approach to variant interpretation uses hierarchical, rule-based classification criteria that take into account evidence from various sources, including population, computational, and predictive data, functional data, segregation data, and other sources. This classification is variant-specific, meaning that the same variant will be classified in the same way for all individuals. However, this approach has some limitations, as illustrated by incomplete penetrance (not all individuals with a pathogenic variant will develop the disease) and variable expressivity (individuals with the same variant can exhibit a wide range of symptoms).

To address these challenges, the present disclosure uses a graph-based approach to personalized interpretation of genetic variants that considers epistasis (genetic interactions) to tailor variant interpretation to an individual's specific genomic background and address issues such as incomplete penetrance, variable expressivity, and variants of uncertain significance (VUS).

Epistasis or genetic interactions refers to the interaction between different genetic factors, where the effect of one gene is dependent on the presence or absence of one or more other genes. This means that the expression of a particular trait (such as a disease) is not solely determined by the presence or absence of a single genetic variant, but rather by the combined effect of multiple genetic factors. Genetic interactions can be either positive, where the presence of one gene enhances the effect of another gene, or negative, where the presence of one gene inhibits or suppresses the effect of another gene.

In the context of personalized interpretation of genetic variants, genetic interactions can play a crucial role in understanding the underlying genetic basis of a particular trait or disease. For example, if an individual has a pathogenic variant in one gene, the presence or absence of other genetic variants in seemingly unrelated genes may affect the likelihood that they will develop the associated disease. In some cases, the presence of certain genetic variants may even completely prevent the expression of a disease, even in the presence of a pathogenic variant in another gene.

Experimental methods to capture genetic interactions have been performed in simple haploid organisms such as yeast by knocking out (e.g., deleting or inactivating) individual genes and pairs of genes, and measuring the impact on phenotype. However, it is not feasible to capture all genetic interactions in complex organisms such as humans experimentally due to the large number of combinations of genes. Machine learning (ML) prediction methods can be used to predict interactions between genes. Although these approaches can be somewhat feasible at the gene level, for instance in humans, having approximately 20,000 to 25,000 genes), the number of potential interactions at the genetic variant level is several orders of magnitude higher than at the gene level. As a result, the number of all possible variant interactions is many orders of magnitude higher and a novel efficient way of modelling these complex interactions is needed, in order to leverage them to improve personalized interpretation of genetic variants on an individual's health and disease.

In the method 100, input data to the system are genetic variants and biomedical domain knowledge (i.e., PPI, Hi-C). The first steps of method 100 can therefore include obtaining the genetic variant profiles of individuals 110 representing the variants in the individual's genome (the individual-specific variant profile), for instance by performing genotyping, exome sequencing or whole genome sequencing, and obtaining a graph representing biomedical domain knowledge 120. Genetic variants are changes to the DNA sequence of an individual that can have various effects on the individual's health and disease status. These changes can range from single nucleotide substitutions to large structural variations, and their effects can range from benign to pathogenic (disease-causing). The biomedical domain knowledge, such as protein-protein-interactions (PPI) and Hi-C, provides valuable context and proximity information for understanding the potential effects of a given variant. The PPI data captures the physical interactions between proteins and provides information about potential genetic interactions among the variants. The Hi-C data captures the proximity of genomic regions in the genome and provides information about the potential impact of variants on the three-dimensional structure of the genome. Both PPI and Hi-C information can be represented graph-theoretically as an edge list including pairs of entities represented as nodes (vertices). For instance, PPI data can include pairs of genes which encode proteins that interact with one another and can therefore be represented for instance as a graph G_{PPI}= [symbol] V_{PPI}, E_{PPI} [symbol] , with V_{PPI} a set of genes and E_{PPI}⊆V_{PPI}² a set of pairs of genes which encode proteins that interact with one another. Hi-C data can include of pairs of genomic loci which have been observed to be in close contact, and can therefore be represented for instance by a graph G_{Hi-C}= [symbol] V_{Hi-C}, E_{Hi-C} [symbol] , with V_{Hi-C} a set of genomic loci and E_{Hi-C}⊆V_{Hi-C}² a set of pairs of genomic loci which have been observed to be in close contact.

The next step 130 can involve generating individual-specific variant graphs, or “patient-specific variant graphs”, each associated with an individual and containing the individual's genetic variants and the dependency information among those genetic variants based on the biomedical domain knowledge. For example, a genetic variant profile associated with a given individual can be provided. The genetic variant profile can be provided as a list of variants and their corresponding genotypes, for example in a VCF (Variant Call Format) file. In some embodiments, the genetic variant profile can be provided in other forms, such as a graph that represents variants in the individual's genome. In such a graph, nodes can correspond to genetic variants of the individual with edges between genetic variants.

An individual-specific variant graph, or patient-specific variant graph, can be constructed by aligning the individual's genetic variant profile with the PPI and Hi-C data. Aligning can for instance include creating edges between the variants based on the potential genetic interactions and proximity information contained in the PPI and Hi-C data, thereby representing known relationships between at least protein-protein interaction relationships and chromosome contact relationships among the variants in the individual's genome. In some embodiments, the individual-specific variant graph can be represented as a list of tuples, such as:

```
[
  (variant_1, variant_5),
  (variant_3, variant_14),
  ...
]
```

For each individual, their individual-specific variant graph can include a list of variant edge pairs. Each variant pair can include of two variants which are connected through PPI or Hi-C data. For instance, if gene A and B are known to interact in the PPI data, then all variants within gene A and B can be connected to each other and added to the list of variant edge pairs.

The resulting individual-specific variant graph which results from the list of variant edge pairs captures the complex dependencies among the variants in an individual's genome and provides valuable information for understanding the effects of a variant on an individual's health and disease status.

After obtaining the individual-specific variant graph for each individual, a subsequent step 140 can include training a graph neural network (GNN) model, such as a graph convolutional network (GCN) model, to perform graph predictions using the “individual-specific variant graph” of each individual in the training dataset. The training process aims to leverage the complex dependencies between genetic variants within each individual-specific variant graph for the purpose of optimizing the prediction output of the GCN for a target phenotype value (i.e., a numerical target value associated with each individual which reflects an observable characteristic of the individual). Examples of phenotype values could be a binary value reflecting whether an individual is affected or unaffected by a disease, or a categorical value reflecting certain predefined disease severity levels, or a continuous value on a continuous rating scale of disease severity of health impacts.

The GCN model can embed one, some or all nodes of one of the graphs defined above in a structure more suited to computation, for instance in a vector. It can be appreciated that a vector embedding encoding information from a graph can be stored using less memory and manipulated by a computer faster and using fewer computational resources than the graph it encodes. Embeddings are vector representations of data that make it easier to perform machine learning on highly sparse inputs with high cardinality (such as genetic variants). In the present case, these embeddings serve to capture the information about the individual variants and relationships between variants. To capture a representation of an individual-specific variant graph which includes multiple nodes, node aggregation can be used to aggregate the node embeddings of the individual nodes. As an example, the aggregation can happen via a reduction step in the model using a mean, sum and/or max over the embeddings (vectors) of the collection of nodes.

Before training the GCN model, the node embeddings can be initialized using either random initial embeddings or predefined embeddings. As examples, predefined embeddings can be obtained as per prior art, e.g., using genetic variants embeddings from pretrained models, such as SNP2Vec, leveraging a denoising autoencoder neural network to encode genetic variants while capturing their similarity and cooccurrence with other variants, or DNABERT, a transformer language model based on DNA sequence, which can be used to encode an embedding representation of the surrounding sequence of a genetic variant, and can be used to provide additional information and context for the variant nodes in the graphs. In some embodiments, the genotype values (i.e., number of copies of the variant present for the individual) can be included as an additional dimension in the node embeddings. In some embodiments, one or more of the chromosome and position, the predicted pathogenicity (e.g., the CADD and/or SIFT scores) and the known pathogenicity classification (e.g., ClinVar) can be included as an additional dimension in the node embeddings. The additional dimensions provide important information about the variant's effect on the individual's health and are included in the variant node embeddings to capture this information.

The initialized node embeddings of the GCN can then be updated during the training process to optimize the prediction output for a target phenotype value. Specifically, a training dataset is created consisting of an individual-specific variant graph for each individual labelled with corresponding target phenotype values (i.e., labels) to be predicted. A differentiable loss function can be defined (e.g., cross-entropy, mean squared error, etc.), and the GCN model is trained through a process of gradient descent and backpropagation. This training process can be performed iteratively for a set number of epochs, during a set duration, and/or until a desired level of evaluation or prediction metric (e.g., accuracy, precision, recall, etc.) value is achieved.

To interpret the contribution of a genetic variant towards the predictions, a method based on counterfactual reasoning can be used at step 150. Broadly described, it can include measuring how the GCN model prediction changes if a variant is removed from the graph of a target individual. More specifically, this can involve first generating an initial individual-specific variant graph for the target individual as described above, the initial individual-specific variant graph representing dependencies among variants in the target individual's genome based on biomedical domain knowledge. Next, the initial individual-specific variant graph can be provided as input to a phenotyping neural network trained as described above, thereby generating a first phenotype prediction 154a based on the initial, “original” graph. Next, the initial individual-specific variant graph can be modified by removing a genetic variant (i.e., removing a variant vertex, which involves removing all pairs of edges which contain the variant vertex being removed), thereby generating a modified individual-specific variant graph. The modified individual-specific variant graph can then be provided as input to the phenotyping neural network to generate a second phenotype prediction 154b based on the modified, “updated” graph. The difference in the prediction before and after removing the variant can then be computed 156 to provide a measure of the variant's contribution to the phenotype. Step 150 can be repeated for each genetic variant of interest to quantify the contribution of each such variant.

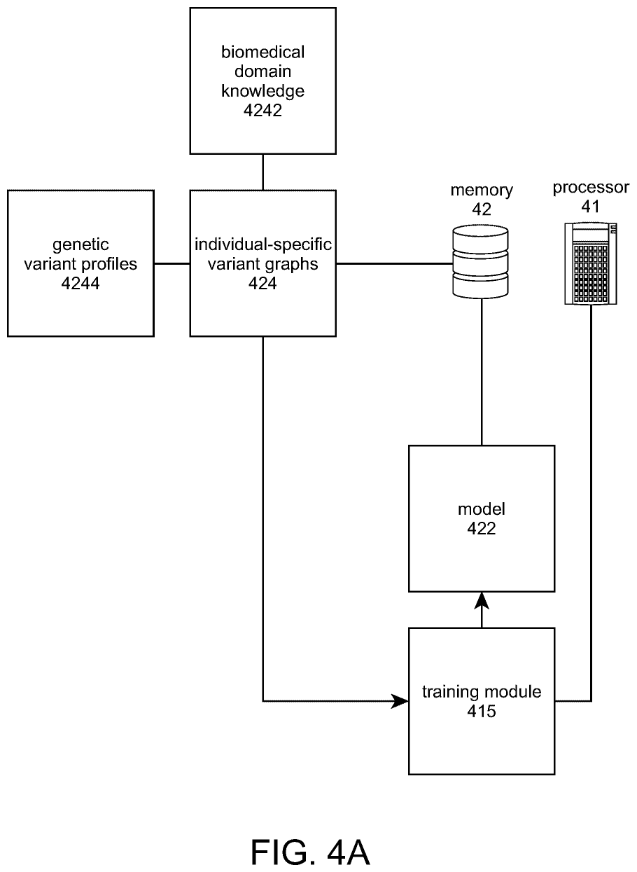

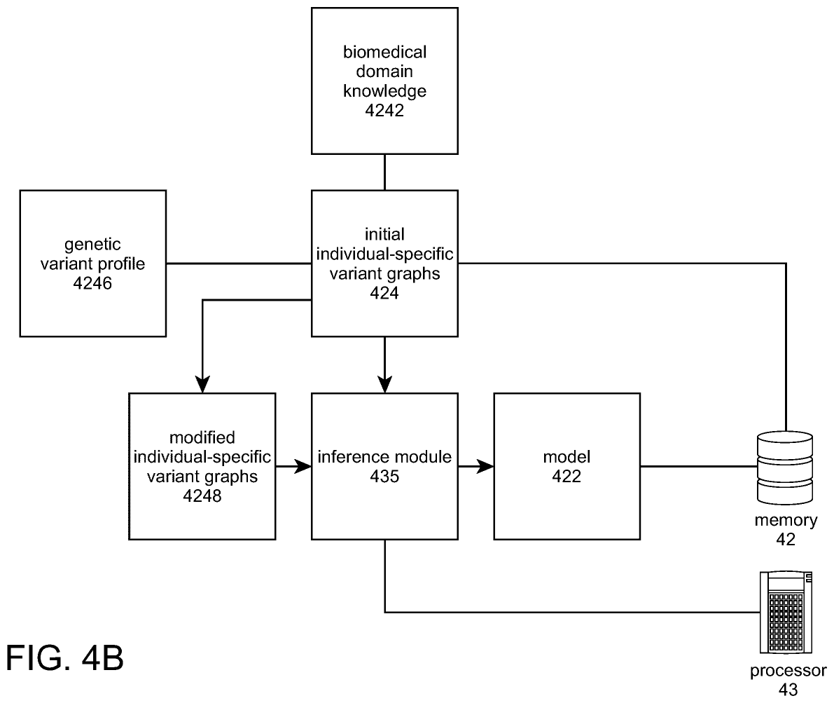

With reference to FIG. 4A, an exemplary system for training a phenotyping neural network is shown. Broadly described, the system includes a processor 41 executing instructions that implement a training module 415 and a memory 42 storing the phenotyping neural network model 422.

The system includes a training module 415 configured to cause the processor 41 to carry out steps for training a phenotyping neural network as described above. For example, the training module 415 can first cause biomedical knowledge 4242 and a number of genetic variant profiles 4244 to be integrated into a corresponding number of individual-specific variant graphs 424, as described above. The individual-specific variant graphs 424 can then be used to train the model 422, as described above.

With reference to FIG. 4B, an exemplary system for obtaining a measure of the contribution of a given genetic variant to the phenotype of an individual using a phenotyping neural network is shown. Broadly described, the system includes a memory 42 storing the treatment prioritization neural network model 422 and the biomedical domain knowledge 4242, and a processor 43 executing instructions that implement an inference module 435.

The system includes an inference module 435 configured to cause the processor 43 to carry out steps for obtaining a measure of the contribution of a given genetic variant to the phenotype of an individual using a phenotyping neural network as described above. For example, the inference module 435 can first cause biomedical knowledge 4242 and the genetic variant profile of a target individual 4246 to be integrated into a corresponding initial individual-specific variant graph 424, as described above. The individual-specific variant graph 424 is suitable for input in the phenotyping neural network model 422. The inference module causes the initial individual-specific variant graph 424 to be modified into a modified individual-specific variant graph 4248 by removing a given genetic variant. The inference module causes both the initial individual-specific variant graph 424 and the modified individual-specific variant graph 4248 to be processed by the phenotyping neural network model 422 to obtain two phenotype predictions, as described above. The two predictions can then be compared to provide a measure of the contribution of the given genetic variant to the phenotype.

It can be appreciated that the systems of FIGS. 4A and 4B can be equivalently implemented as one or a plurality of systems, and that processors 41 and 43 can be the same processor or can be different processors. Moreover, it can be appreciated that each of processors 41 and 43 can correspond to a plurality of processors implementing the training module 415 and/or the inference module 435, for instance for parallel and/or distributed processing.

The method described above uses a graph representation to capture the complex interactions among genetic variants in an individual's genome. This enables a system implementing the method to better decipher the effects of a variant in the context of an individual's genomic background and address challenges such as incomplete penetrance and variable expressivity for single genes and multifactorial diseases. Such a system provides a more comprehensive and personalized understanding of the potential effects of genetic variants on an individual's health.

An implementation of a system using the method described above as described above was made to demonstrate the potential of the proposed system for the personalized interpretation of genetic variant effects based on the phenotype of cardiovascular disease status. Variants derived from exome sequencing data from individuals were combined with open-source PPI and chromatin contact data to generate individual-specific variant graphs for subsequent analysis using the graph neural network approach described before.

Exome variants were selected to be classified as high or moderate impact by the variant effect predictor (VEP) tool from Ensembl, described in MCLAREN, William, GIL, Laurent, HUNT, Sarah E., et al.; The ensembl variant effect predictor; Genome biology, 2016, vol. 17, no 1, p. 1-14, the disclosure of which is hereby incorporated by reference in its entirety. VEP simply assigns a predefined impact classification based on the consequence of each variant on a gene (e.g., missense, stop gained, stop lost, frameshift, etc.). Poor quality variants with greater than 1% missing genotype calls were removed. In total, 4,823,277 exome variants were included for subsequent analysis.

Next, the individual-specific variant graphs were constructed. For chromatin contact data, the contact matrix file derived from MicroC experiments with HFFc6 (human foreskin fibroblast cell line) described in KRIETENSTEIN, Nils, ABRAHAM, Sameer, VENEV, Sergey V., et al.; Ultrastructural details of mammalian chromosome architecture; Molecular cell, 2020, vol. 78, no 3, p. 554-565, the disclosure of which is hereby incorporated by reference in its entirety, was used as the data source. All genetic loci (at 1000 base pair resolution) in the contact matrix file that corresponded to at least one of the filtered exome variants were retained. Next, all observed contact pairs between each of the filtered genetic loci were extracted. For PPI, for all interactions with a score greater than 500 from the STRING database described in SZKLARCZYK, Damian, GABLE, Annika L., NASTOU, Katerina C., et al.; The STRING database in 2021: customizable protein-protein networks, and functional characterization of user-uploaded gene/measurement sets; Nucleic acids research, 2021, vol. 49, no D1, p. D605-D612, the disclosure of which is hereby incorporated by reference in its entirety, were retained. For each individual from the exome dataset, a graph consisting of all of the filtered variants of the individual with edges between the variants created based on the chromatin contact and PPI information was generated.

The experimentation explored cardiovascular disease (CVD), which was defined based on the occurrence of at least one of the following events: coronary and carotid revascularization, myocardial infarction, ischemic stroke. Next, the described system based on graph neural networks was used to model the contribution of genetic variants to the CVD phenotype. For comparison, a logistic regression model with L2-regularization (weight of 0.1) and a dense neural network model were used.

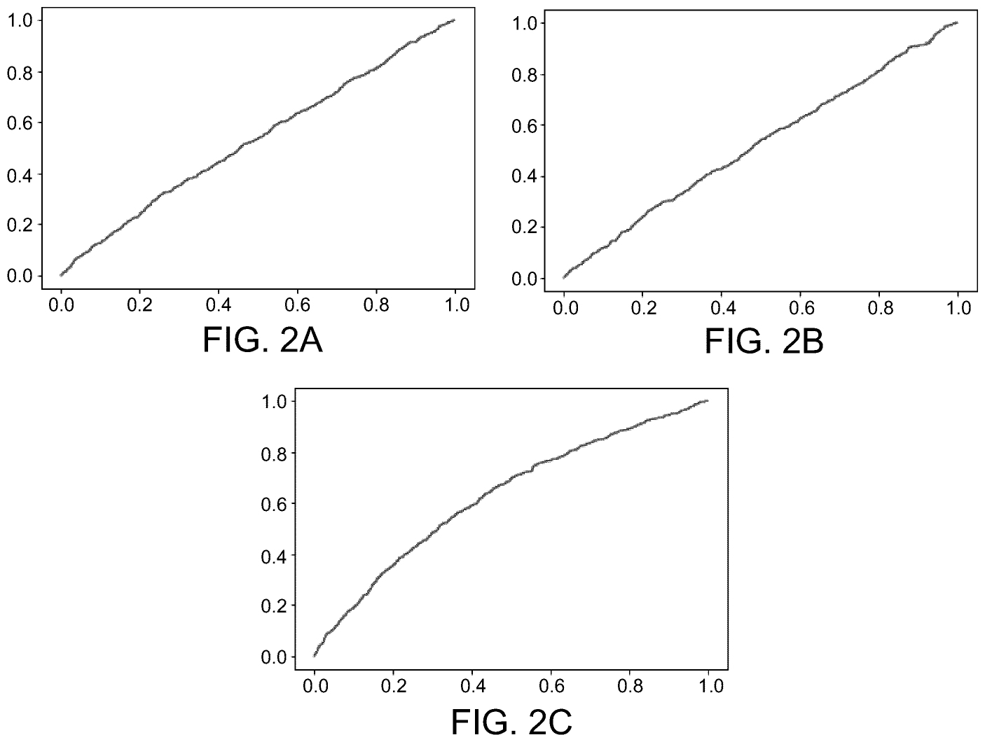

The training dataset consisted of 50,000 samples while the validation and testing dataset consisted of 10,000 samples each. The training and validation datasets were used to perform cross-validation to prevent overfitting and determine the most optimal model for use during evaluation. The evaluation metric, applied to the testing dataset, is the area under the receiver operating characteristic curve (AUC). FIGS. 2A, 2B, and 2C show the AUC performance of the logistic regression model, dense neural network model, and graph neural network model respectively. The logistic regression and dense neural network models performed similarly at AUCs of 0.53 and 0.52 respectively. The graph neural network model, at 0.63, shows a notable 18.9% improvement over the logistic regression model. The improvement in prediction performance suggests that the approach disclosed herein was able to effectively leverage the dependency information among genetic variants represented by the variant-variant graph structure.

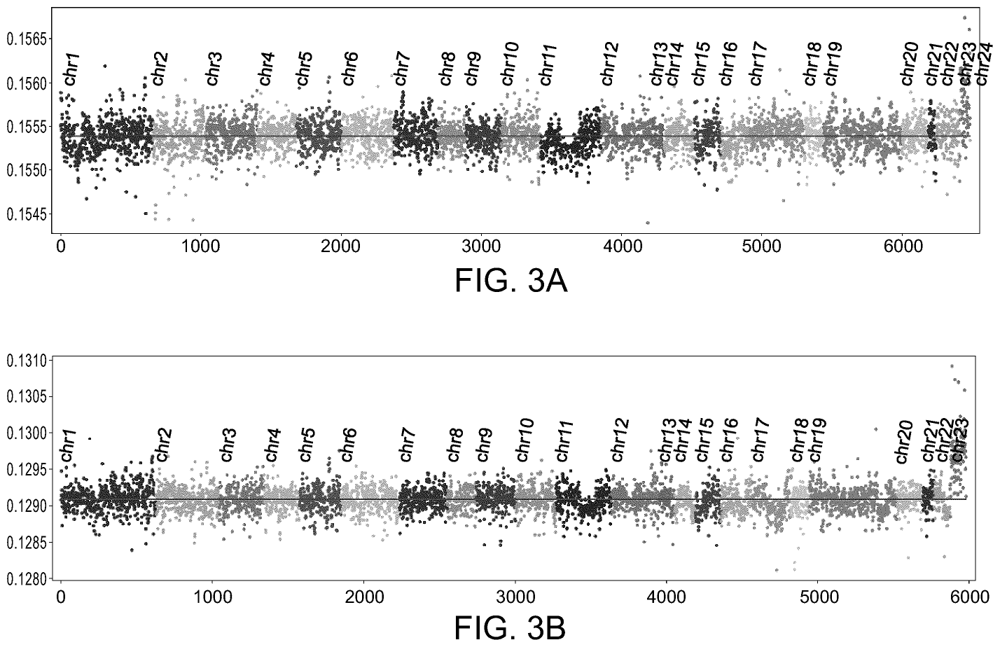

Next, the use of the counterfactual method of variant interpretation based on the graph neural network model was demonstrated. Using the counterfactual method, it is possible examine the individual contributions of each variant to the overall predicted likelihood of CVD. By comparing the predicted likelihood with and without each variant, one can determine the effect of each variant on disease risk. FIGS. 3A and 3B show examples of this for two individuals “A” (FIG. 3A) and “B” (FIG. 3B) with CVD who also had the highest predicted likelihood of CVD. Variants which increase the predicted likelihood of disease (relative to the original prediction, represented by the horizontal line) are shown as dots below the original prediction line. The variants which decrease the risk predicted likelihood of disease when present are shown as dots above the original prediction line. Variants are associated with the chromosome they are present in and ordered by their location on the chromosome. Notably, for individuals A and B there appear to be a cluster of variants in chromosome 11 which appear to significantly increase the risk of CVD for the given individual. For individual B, there appear to be an additional cluster of variants in chromosome 17 which increases the risk of CVD which is not present in individual A. Furthermore, the relative protective effects of variants on chromosome X (chromosome 23) for individual A appear to have a much higher protective effect compared individual B.

In conclusion, it was demonstrated the counterfactual method and the graph neural network model to be a useful tool for personalized variant interpretation which can help inform the development of personalized prevention and treatment strategies for diseases like CVD.

In the description presented above, a system and method for genetic variant interpretation, which includes the creation and use of “individual-specific variant graphs” was provided. The described approach uses graph representations of genetic variants based on biomedical domain knowledge, such as protein-protein-interaction (PPI) and chromosome contact (Hi-C) information. The approach makes use of individual-specific variant graphs (i.e. a different genetic variant graph for each individual), which are used as the input to a GCN model for each prediction instance. The method begins with creating individual-specific variant graphs based on the genetic variant genotypes of each patient. Next, a GCN is created for the purpose of graph prediction (i.e., making predictions based on the input individual-specific variant graphs). Next, supervised model training is performed where a given target label (e.g. health or disease status) is predicted. Finally, after a model is trained, counterfactual reasoning approach is applied to obtain the contribution of each genetic variant to a prediction output.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A method for phenotyping, the method comprising:
generating a plurality of individual-specific variant graphs, each individual-specific variant graph being associated with an individual and representing dependencies among variants in a genome of the individual based on biomedical domain knowledge;
generating a training dataset comprising at least some of the plurality of individual-specific variants graphs labelled according to corresponding target phenotype values to be predicted; and
training a phenotyping neural network model using the training dataset to generate a phenotype prediction from an input individual-specific variant graph.

2. The method of claim 1, wherein each individual-specific variant graph represents known relationships between at least one of protein-protein interactions and chromosome contact among the variants in the genome of the individual.

3. The method of claim 2, wherein each individual-specific variant graph comprises a plurality of variant pairs, each variant pair comprising two variants connected through a protein-protein interaction relationship and/or a chromosome contact relationship.

4. The method of claim 2, wherein generating the plurality of individual-specific variant graphs comprises, for each individual:
providing a genetic variant profile representing the variants in the genome of the individual;
providing the biomedical domain knowledge as an edge list comprising pairs of genes which encode proteins that interact with one another and/or pairs of genomic loci which have been observed to be in close contact; and
generating an individual-specific variant graph by aligning the genetic variant profile with the biomedical domain knowledge by creating variant pairs in the individual-specific variant graph corresponding to the pairs of genes of the edge list and/or the pairs of genomic loci of the edge list.

5. The method of claim 1, wherein the target phenotype values comprise a binary value reflecting a status of whether an individual is affected or unaffected by a disease.

6. The method of claim 1, wherein the target phenotype values comprise a categorical value representing a plurality of predefined disease severity levels, or a continuous value representing a rating of disease severity or health impact.

7. The method of claim 1, wherein the phenotyping neural network model is a graph neural network model.

8. The method of claim 1, wherein training the phenotyping neural network model comprises embedding at least a subset of nodes of the individual-specific variants graphs in vectors, wherein the embeddings comprise at least one of: a number of copies of a variant present in the individual, one or more of a chromosome and position, a predicted pathogenicity, and a known pathogenicity classification.

9. The method of claim 8, wherein the embeddings are updated during the training of the phenotyping neural network model to optimize prediction output for a target phenotype value.

10. The method of claim 1, further comprising:
generating an initial individual-specific variant graph for a target individual, the initial individual-specific variant graph representing dependencies among variants in a genome of the target individual based on the biomedical domain knowledge;
providing the initial individual-specific variant graph as input to the phenotyping neural network model, thereby generating a first phenotype prediction;
modifying the initial individual-specific variant graph to remove a genetic variant, thereby generating a modified individual-specific variant graph;
providing the modified individual-specific variant graph as input to the phenotyping neural network model, thereby generating a second phenotype prediction; and
calculating a difference between the first phenotype prediction and the second phenotype prediction, the difference providing a measure of a contribution of the genetic variant to a phenotype of the target individual.

11. A system for phenotyping, the system comprising:
one or more processor;
memory having a phenotyping neural network model stored thereon; and
a training module configured to cause the one or more processor to:
generate a plurality of individual-specific variant graphs, each individual-specific variant graph being associated with an individual and representing dependencies among variants in a genome of the individual based on biomedical domain knowledge;
generate a training dataset comprising at least some of the plurality of individual-specific variants graphs labelled according to corresponding target phenotype values to be predicted; and
train the phenotyping neural network model using the training dataset to generate a phenotype prediction from an input individual-specific variant graph.

12. The system of claim 11, wherein each individual-specific variant graph represents known relationships between at least one of protein-protein interactions and chromosome contact among the variants in the genome of the individual.

13. The system of claim 12, wherein each individual-specific variant graph comprises a plurality of variant pairs, each variant pair comprising two variants connected through a protein-protein interaction relationship and/or a chromosome contact relationship.

14. The system of claim 12, wherein generating the plurality of individual-specific variant graphs comprises, for each individual, causing the one or more processor to:
provide a genetic variant profile representing the variants in the genome of the individual;
provide the biomedical domain knowledge as an edge list comprising pairs of genes which encode proteins that interact with one another and/or pairs of genomic loci which have been observed to be in close contact; and
generate an individual-specific variant graph by aligning the genetic variant profile with the biomedical domain knowledge by creating variant pairs in the individual-specific variant graph corresponding to the pairs of genes of the edge list and/or the pairs of genomic loci of the edge list.

15. The system of claim 11, wherein the target phenotype values comprise a binary value reflecting a status of whether an individual is affected or unaffected by a disease.

16. The system of claim 11, wherein the target phenotype values comprise a categorical value representing a plurality of predefined disease severity levels, or a continuous value representing a rating of disease severity or health impact.

17. The system of claim 11, wherein the phenotyping neural network model is a graph neural network model.

18. The system of claim 11, wherein training the phenotyping neural network model comprises causing the one or more processor to embed at least a subset of nodes of the individual-specific variants graphs in vectors, wherein the embeddings comprise at least one of: a number of copies of a variant present in the individual, one or more of a chromosome and position, a predicted pathogenicity, and a known pathogenicity classification.

19. The system of claim 18, wherein the embeddings are updated during the training of the phenotyping neural network model to optimize prediction output for a target phenotype value.

20. A system for phenotyping, the system comprising:
one or more processor;
memory having a phenotyping neural network model stored thereon; and
an inference module configured to cause the one or more processor to:
generate an initial individual-specific variant graph for a target individual, the initial individual-specific variant graph representing dependencies among variants in a genome of the target individual based on biomedical domain knowledge;
provide the initial individual-specific variant graph as input to the phenotyping neural network model, thereby generating a first phenotype prediction;
modify the initial individual-specific variant graph to remove a genetic variant, thereby generating a modified individual-specific variant graph;
provide the modified individual-specific variant graph as input to the phenotyping neural network model, thereby generating a second phenotype prediction; and
calculate a difference between the first phenotype prediction and the second phenotype prediction, the difference providing a measure of a contribution of the genetic variant to a phenotype of the target individual.
